(19) United States
(12) Patent Application Publication
(10) Pub. No.: US 2017/0143355 A1
(43) Pub. Date: May 25, 2017

(54) Path Creation Through Occlusion

(71) Applicants: William Nicholson, (US); Vascular Solutions, Inc., Minneapolis, MN (US)

(72) Inventors: William Nicholson, York, PA (US); Chad Kugler, Buffalo, MN (US); Joshua Brenizer, Maple Grove, MN (US); Howard Root, Excelsior, MN (US)

(21) Appl. No.: 15/254,386

(22) Filed: Sep. 1, 2016

Related U.S. Application Data
(60) Provisional application No. 62/257,777, filed on Nov. 20, 2015.

(51) Int. Cl. A61B 17/22

(52) U.S. Cl. CPC A61B 17/22; A61B 2017/22045; A61B 2017/22072; A61B 2017/22094; A61B 2017/22001

(57) ABSTRACT

This patent document discloses assemblies, alignment devices and methods for creating a bi-directional path through an occlusion. An alignment device can include a positioning member and a tubular member. The tubular member can be coupled with a distal end portion of the positioning member and can include proximal and distal progressive size-changing shapes separated by a narrow neck passageway. An assembly can include the alignment device, a guide catheter having a lumen in which the alignment device is at least partially positioned, a guidewire, and a specialty catheter for supporting the guidewire. The alignment device and the guide catheter can be advanced in an antegrade direction to a proximal end of the occlusion, and the guidewire and the specialty catheter can be advanced in a retrograde direction to a distal end of the occlusion. The guidewire and the specialty catheter can be manipulated through the occlusion and into the distal end of the tubular member.

### CLAIM OF PRIORITY

This non-provisional patent document claims the benefit of priority under 35 U.S.C. §119(e) to U.S. Provisional Patent Application Ser. No. 62/257,777, entitled “PATH CREATION THROUGH OCCLUSION” and filed on Nov. 20, 2015, which is herein incorporated by reference in its entirety.

### TECHNICAL FIELD

This patent document relates to the field of surgical instruments for use in treating severe or chronic total occlusions of blood vessels. More particularly, but not by way of limitation, the patent document relates to the penetration of hard plaque that partially or completely occludes a blood vessel and disturbs blood flow through such vessel.

### BACKGROUND

Atherosclerosis is a complex, progressive and degenerative condition resulting in the build-up of cholesterol and other occlusive materials, known as plaque, on the walls of blood vessels. The accumulation of plaque narrows the interior or lumen of the vessels reducing blood flow. Plaque occurs in blood vessels in several different forms and can be located in many different anatomies throughout a vascular system. Plaque can vary in composition, with portions that are hard and fibrous, known as calcified plaque, and other portions that are soft and fatty.

Over time, plaque deposits can become large enough to substantially reduce or totally occlude blood flow through a vessel, which can lead to symptoms associated with low blood flow, including cardiac arrest, stroke, or tissue or organ necrosis. Chronic Total Occlusions (CTOs) are one type of plaque deposit, usually including calcified plaque portions, which block the blood path through the affected vessel. To treat plaque deposits and improve or resolve low blood flow symptoms, it is desirable to restore or improve blood flow through the affected vessel.

A common procedure for treating plaque deposits is percutaneous transluminal angioplasty. During an angioplasty procedure, access to a desired blood vessel is obtained and a guidewire is introduced into the blood vessel in an antegrade direction. The guidewire is maneuvered into place by being passed into and through the occlusion and acts as a rail for positioning a subsequent treatment device, such as a dilatation balloon catheter. When appropriately positioned within the occlusion, the dilatation balloon catheter can be inflated to apply radial pressure and compress the plaque deposit to increase blood flow through the affected vessel.

### OVERVIEW

Percutaneous treatment of CTOs can be challenging. A failure mode in the treatment of CTOs is an inability to successfully pass a guidewire in an antegrade direction across the occlusion and into the true lumen of the blood vessel distal to the occlusion. The occlusion can be composed of calcified plaque having a hard, dense proximal cap that prevents penetration by the guidewire. The present inventors recognize that approaching the occlusion from its softer, less dense distal end can allow successful passage of the guidewire into and through the occlusion. After crossing the occlusion, the retrograde-advanced guidewire can be captured and subsequently exchanged with a different guidewire having properties advantageous for positioning within the vessel or for guiding one or more treatment devices within or through the occlusion. The present inventors further recognize the potential complications for capturing the retrograde guidewire on the proximal side of the occlusion and the extra time and cost of externalizing the guidewire and inserting a specialty catheter over it to effectuate a guidewire exchange. It is these recognitions that led to the present assemblies, alignment devices and methods for creating a bi-directional path through an occlusion.

An alignment device can include a positioning member and a tubular member. The tubular member can be coupled with a distal end portion of the positioning member and can include proximal and distal bi-directional, progressive size-changing shapes (e.g., funnels) separated by a narrow neck passageway. An assembly can include the alignment device, a guide catheter having a lumen in which the alignment device is at least partially positioned, a guidewire, and a specialty catheter for supporting the guidewire. The alignment device and the guide catheter can be advanced in an antegrade direction to a proximal end of the occlusion, and the guidewire and the specialty catheter can be advanced in a retrograde direction to a distal end of the occlusion. The guidewire and the specialty catheter can be manipulated in the retrograde direction through the occlusion and into the distal progressive size-changing shape of the tubular member, thereby aligning a lumen of the specialty catheter and the narrow neck passageway of the tubular member. After withdrawing the guidewire, a second guidewire can be advanced through the lumen of the guide catheter and passageway of the tubular member and into the lumen of the specialty catheter to a position distal to the occlusion. The second guidewire can then be used to guide one or more treatment devices within or through the occlusion.

A method for creating a bi-directional path through an occlusion can include introducing a guide catheter into a blood vessel at a location upstream of the occlusion, and advancing the guide catheter in an antegrade direction toward the occlusion's proximal end. An alignment device, including a positioning member and a tubular member having proximal and distal bi-directional, progressive size-changing shapes separated by a narrow neck passageway, can be introduced into a proximal end portion of the guide catheter and advanced to or partially beyond the guide catheter's distal end portion, where the proximal progressive size-changing shape can engage against an inner surface of the guide catheter and the distal progressive size-changing shape can engage against the inner surface of the guide catheter or an adjacent luminal wall of the blood vessel. A guidewire and a specialty catheter can be introduced into the blood vessel at a location downstream of the occlusion and advanced in a retrograde direction toward the occlusion's distal end. With the guidewire leading the way, the guidewire and the specialty catheter can be manipulated through the occlusion and into the distal progressive size-changing shape of the tubular member. The docking of the specialty catheter's distal tip into the distal progressive size-changing shape can align the lumen of the specialty catheter and the narrow neck passageway of the tubular member.

These and other examples and features of the present assemblies, alignment devices and methods will be set forth, at least in part, in the following Detailed Description. This Overview is intended to provide non-limiting examples of the present subject matter—it is not intended to provide an exclusive or exhaustive explanation. The Detailed Description below is included to provide further information about the present assemblies, alignment devices and methods.

### BRIEF DESCRIPTION OF THE DRAWINGS

In the drawings, like numerals can be used to describe similar features and components throughout the several views. The drawings illustrate generally, by way of example but not by way of limitation, various embodiments discussed in the present patent document.

FIG. 1 illustrates a schematic view of coronary anatomy and an occlusion located within a coronary vessel.

FIG. 2 illustrates a close-up view of an occlusion in the form of a CTO.

FIG. 3 illustrates a schematic view of a guidewire and a specialty catheter advanced in a retrograde direction toward a distal end of the occlusion of FIG. 2.

FIG. 4 illustrates a schematic view of a first guide catheter, a guidewire and a specialty catheter advanced in a retrograde direction toward a distal end of the occlusion of FIG. 2, and a second guide catheter and an alignment device advanced in an antegrade toward a proximal end of this occlusion.

FIGS. 5A-D illustrate sequential views of a guidewire and a specialty catheter advanced in a retrograde direction through an occlusion and captured by an alignment device, as constructed in accordance with at least one embodiment.

FIG. 6 illustrates a schematic view of an alignment device for capturing a first guidewire and a specialty catheter advanced in a retrograde direction, and for subsequently guiding a second guidewire advanced in an antegrade direction, as constructed in accordance with at least one embodiment.

FIG. 7 illustrates a schematic view of an alignment device for capturing a first guidewire and a specialty catheter advanced in a retrograde direction, and for subsequently guiding a second guidewire advanced in an antegrade direction, as constructed in accordance with at least one other embodiment.

FIG. 8 illustrates a method of using an alignment device for creating a bi-directional path through an occlusion, as constructed in accordance with at least one embodiment.

The drawing figures are not necessarily to scale. Certain features and components may be shown exaggerated in scale or in schematic form and some details may not be shown in the interest of clarity and conciseness.

### DETAILED DESCRIPTION

The present subject matter provides assemblies, alignment devices and methods for creating a bi-directional path through an occlusion, particularly CTOs, using a combined antegrade and retrograde approach. A distal end of the occlusion can be penetrated using a retrograde guidewire supported by a specialty catheter. A progressive size-changing shape of an alignment device located within or partially beyond a distal end portion of a guide catheter can be positioned at a proximal end of the occlusion. After being maneuvered through the occlusion, distal end portions of the guidewire and the specialty catheter can be captured by the progressive size-changing shape and a bi-directional path through the occlusion can be created by the serial positioning of lumens in the guide catheter, the alignment device and the specialty catheter.

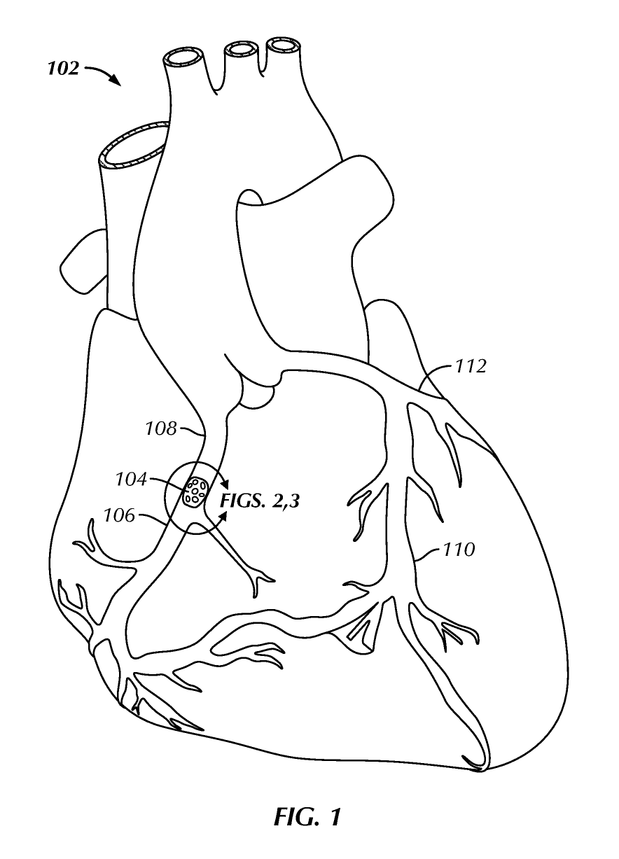

FIG. 1 illustrates a schematic view of coronary anatomy 102 and an occlusion 104 located within a blood vessel 106. The blood vessel 106 shown is a coronary vessel, but it can be any vessel in which blood flows through the hollow tubular cavity. The occlusion 104 within the blood vessel 106 obstructs the flow of blood and can have fatal consequences. The occlusion 104 shown is a CTO, which, when occurring in the coronary vessels, most often occurs in the right coronary artery 108, the distal left anterior descending artery 110, or the circumflex 112.

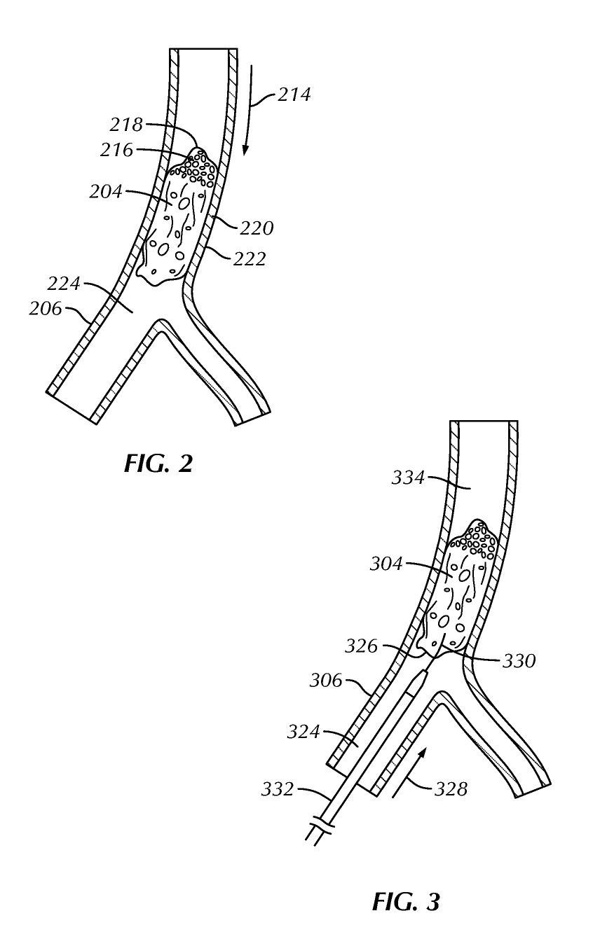

One method of recanalizing an occlusion 204 is by using techniques in which a guidewire penetrates the occlusion and subsequently a treatment device (e.g., a dilatation balloon catheter) guided over the guidewire recanalizes the vessel 206. Typically, the guidewire approaches the occlusion 204 from an antegrade direction 214 (i.e., in the direction of blood flow). Depending on the type and composition of the occlusion 204, it may be difficult to successfully penetrate the occlusion using standard guidewire techniques alone. For example, as shown in FIG. 2, a cap 216 at a proximal end 218 of the occlusion 204 may be composed of dense, fibrous tissue (e.g., calcified plaque) that does not allow the guidewire to pass. The difficulties in penetrating the cap 216 of the occlusion 204 can lead to the guidewire slipping away from the surface of the cap and entering into the vessel's subintimal space 220. The penetration of the subintimal space 220 can lead to the puncturing of the luminal wall 222 of the blood vessel 206, which may cause bleeding as well as other undesirable side effects. Furthermore, by penetrating the subintimal space 220 instead of the cap 216, it can be substantially more difficult for a catheter to advance into the distal true lumen 224 of the vessel to complete the recanalization.

FIG. 3 illustrates that by approaching an occlusion 304 within a blood vessel 306 from its less dense distal end 326 and in a retrograde direction 328 (i.e., against the flow of blood), a guidewire 330 can be successfully passed into and through the occlusion. The guidewire 330 can be placed at the distal end 326 of the occlusion 304 and then advanced into the occlusion from a distal true lumen 324 of the blood vessel with the help of a specialty catheter 332. The specialty catheter 332 can provide support to the guidewire 330. The guidewire 330 and the specialty catheter 332 can be maneuvered through the occlusion 304 and create a continuous channel from the distal true lumen 324 to a proximal true lumen 334 of the blood vessel 306.

After crossing the occlusion, existing retrograde techniques dictate that the guidewire be captured, externalized and exchanged for another guidewire having properties more suitable (e.g., having greater flexibility) for positioning within the blood vessel or for guiding an over-the-wire treatment device. During retrograde procedures, retrieving and exchanging guidewires from the proximal end of the occlusion can be difficult, time-consuming and frustrating for the treating clinician. Manipulation of guidewire retrieval snares, for example, can be inherently dangerous (e.g., can lead to luminal wall skiving or perforation), unreliable, damaging to the guidewire, and time-consuming. Further, existing retrograde techniques require the use of long guidewires having a length of about 300 centimeters (cm) or more, which allows the guidewires to be externalized at both ends but which can be difficult for the treating clinician to control and handle. Once the guidewire is finally captured and externalized, a specialty catheter can be advanced in an antegrade direction over the guidewire to a position past the occlusion. The guidewire can then be removed and another guidewire can be introduced through a lumen of the specialty catheter.

The present assemblies, alignment devices and methods improve upon existing retrograde techniques, specifically guidewire capture and exchange techniques. The assemblies, alignment devices and methods create a bi-directional path through the occlusion that allows the use of shorter guidewire lengths (e.g., guidewires having a length of 175 cm or less) providing more control over the guidewire. The assemblies, alignment devices and method also eliminate the need for externalizing the guidewire and inserting a specialty catheter to effectuate a guidewire exchange, thereby reducing procedure times and medical waste.

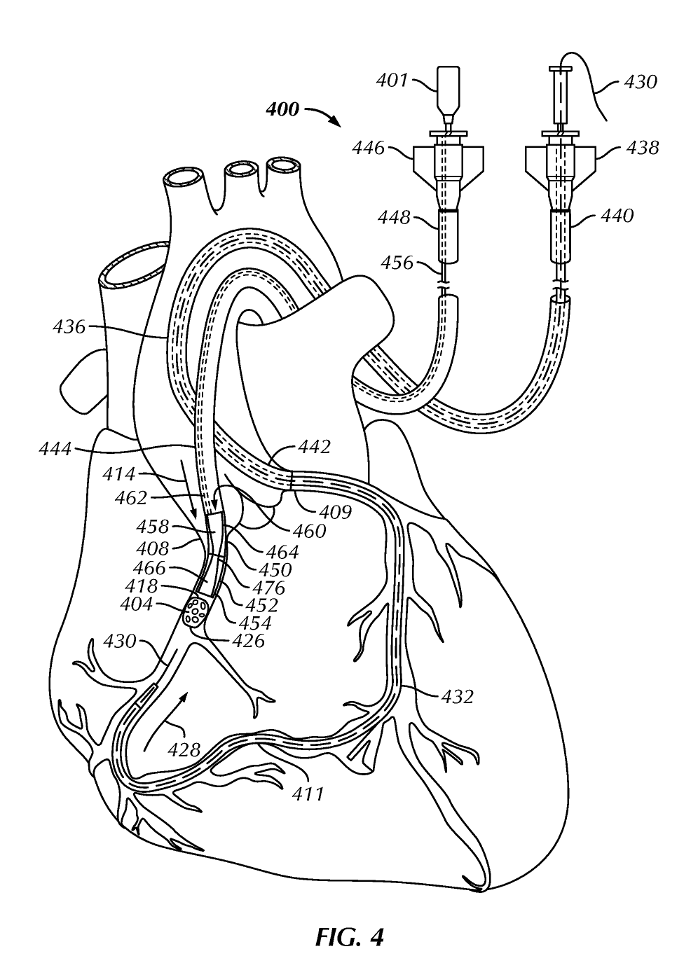

FIG. 4 illustrates a schematic view of an assembly 400 for creating a bi-directional path through an occlusion 404 located in a right coronary artery 408. A short guidewire 430 and a specialty catheter 432 can be introduced into a first guide catheter 436 having a valve 438 attached to its proximal end 440 and having its distal end 442 positioned in a left coronary artery 409. The guidewire 430 and specialty catheter 432 can be advanced in a retrograde direction 428 through the first guide catheter 436 and one or more distal collateral vessels 411 and can approach the occlusion 404 from its distal end 426. An alignment device 401 can be introduced into a second guide catheter 444 having a valve 446 attached to its proximal end 448 and having its distal end 450 positioned in the right coronary artery 408 proximal to the occlusion 404. The alignment device 401 can be advanced in an antegrade direction 414 to or partially beyond the distal end 450 of the second guide catheter 444. Optionally, a guide extension catheter 452 can be advanced through the second guide catheter 444 to achieve a guide catheter position closer to the proximal end 418 of the occlusion 404. In this optional scenario, the alignment device 401 can be advanced in an antegrade direction 414 to or partially beyond the distal end 454 of the guide extension catheter 452.

As discussed further below, the alignment device 401 can include a positioning member 456 and a tubular member 458 defining a passageway 460. A distal end portion 462 of the positioning member 456 can be coupled with the tubular member 458 and can extend proximally therefrom for slidably positioning the tubular member within or partially beyond the second guide catheter 444 or the guide extension catheter 452. A proximal end portion 464 and a distal end portion 466 of the tubular member 458 can include a progressive size-changing shape such as a funnel. Each funnel can have an opening with a diameter greater than a diameter of an intermediate portion 476 of the tubular member 458, and each funnel's outer surface can be engageable with an inner surface of the second guide catheter 444 or the guide extension catheter 452 or an adjacent luminal wall of the right coronary artery 408. The funnel at the proximal end portion 464 of the tubular member 458 can define a narrowing (converging) portion of the defined passageway 460 in a proximal-to-distal direction, while the funnel at the distal end portion 466 of the tubular member 458 can define an enlarging (diverging) portion of the passageway 460 in the proximal-to-distal direction.

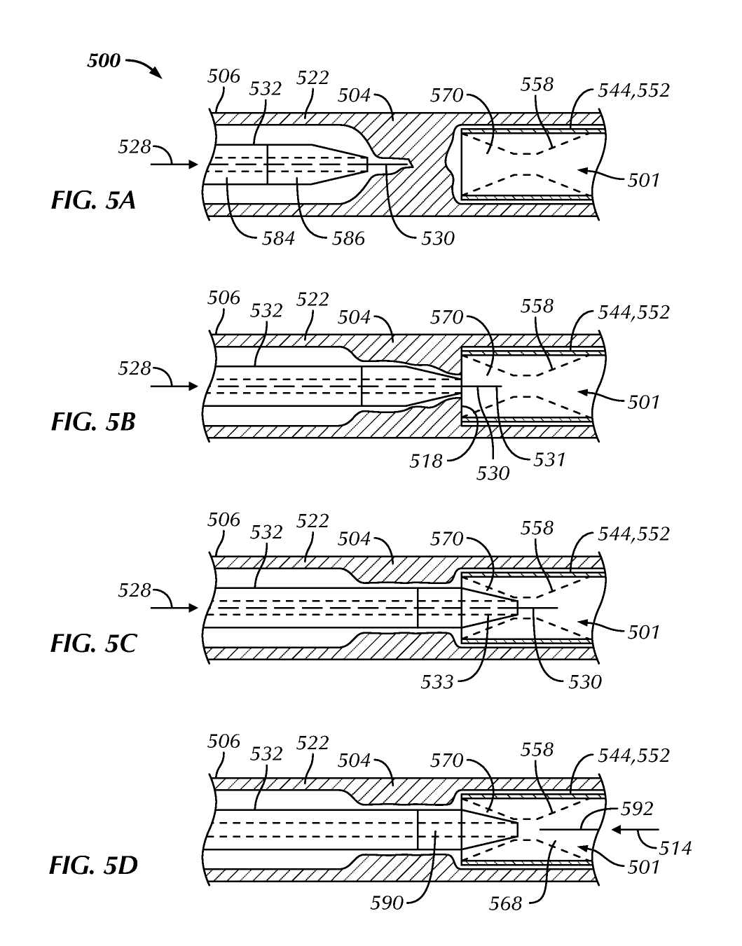

FIGS. 5A-5D illustrate sequential views of a guidewire 530 and a specialty catheter 532 being advanced in a retrograde direction 528 through an occlusion 504 and captured by a distal funnel 570 of the alignment device 501, which is shown positioned within a second guide catheter 544 or a guide extension catheter 552 but which can alternatively extend partially beyond the second guide catheter 544 or the guide extension catheter 552 and engage an adjacent luminal wall of a blood vessel 506.

In FIG. 5A, the guidewire 530 and the specialty catheter 532 are incrementally advanced in the retrograde direction 528 through the occlusion 504. The guidewire 530 is advanced ahead of the specialty catheter 532, which can provide column and/or torque support to the guidewire 530 as a treating clinician asserts a pushing or rotating force to the guidewire or specialty catheter's proximal end. The specialty catheter 532 can include an elongate shaft body 584 and a tip member 586 disposed at a distal end of the shaft body. The tip member 586 can be made from a metal or a polymer and can include one or more helical threads around its outer surface or a sharpened or tapered tip to facilitate advancement through the occlusion 504.

When the distal ends 531, 533 of the guidewire 530 and the specialty catheter 532 emerge from the proximal end 518 of the occlusion 504, each can be captured by the enlarged target opening of the distal funnel 570 of the tubular member 558. FIG. 5B illustrates the distal end 531 of the guidewire 530 emerging from the occlusion 504 and being funneled into the passageway of the tubular member 558. Using the guidewire 530 as a rail, the specialty catheter 532 can subsequently emerge from the occlusion 504 and its distal end 533 can be guided into a mating arrangement with the distal funnel 570, as illustrated in FIG. 5C. In this way, the assembly 500 allows for the reliable capture of the retrograde guidewire 530 and specialty catheter 532 without damaging the components or the luminal wall 522 of the blood vessel 506.

With the distal end 533 of the specialty catheter 532 docked in the distal funnel 570 of the tubular member 558, a bi-directional path 590 is created through the occlusion 504. In an antegrade direction 514, the bi-directional path 590 is created by the serial positioning of lumens of the second guide catheter 544 and optionally the guide extension catheter 552, the passageway of the tubular member 558, and a lumen of the specialty catheter 532. In the retrograde direction 528, the bi-directional path 590 is created by the serial positioning of the lumen of the specialty catheter 532, the passageway of the tubular member 558, and the lumens of the optional guide extension catheter 552 and the second guide catheter 544. By way of example, the path 590 allows a treatment procedure to be continued in the antegrade direction 514 from the upstream (or proximal) end 518 of the occlusion 504 without having to capture and externalize the distal end 531 of the guidewire 530. Instead, as illustrated in FIG. 5D, the guidewire 531 can be removed from a (downstream) first guide catheter, and a second guidewire 592 can be introduced into the (upstream) second guide catheter 544 and advanced through a proximal funnel 568 and passageway of the tubular member 558 and into the lumen of the specialty catheter 532 to a position within or distal to (beyond) the occlusion 504.

At this time, the specialty catheter 532, the first guide catheter, and the alignment device 501 can be removed and the occlusion 504 can be treated in a conventional manner. For example, a dilatation balloon catheter can be introduced into the second guide catheter 544 and advanced over the second guidewire 592 to a position in which its balloon is within the occlusion 504. The balloon can be inflated, separating or fracturing the occlusion 504, and a stent can subsequently be placed within the occlusion 504.

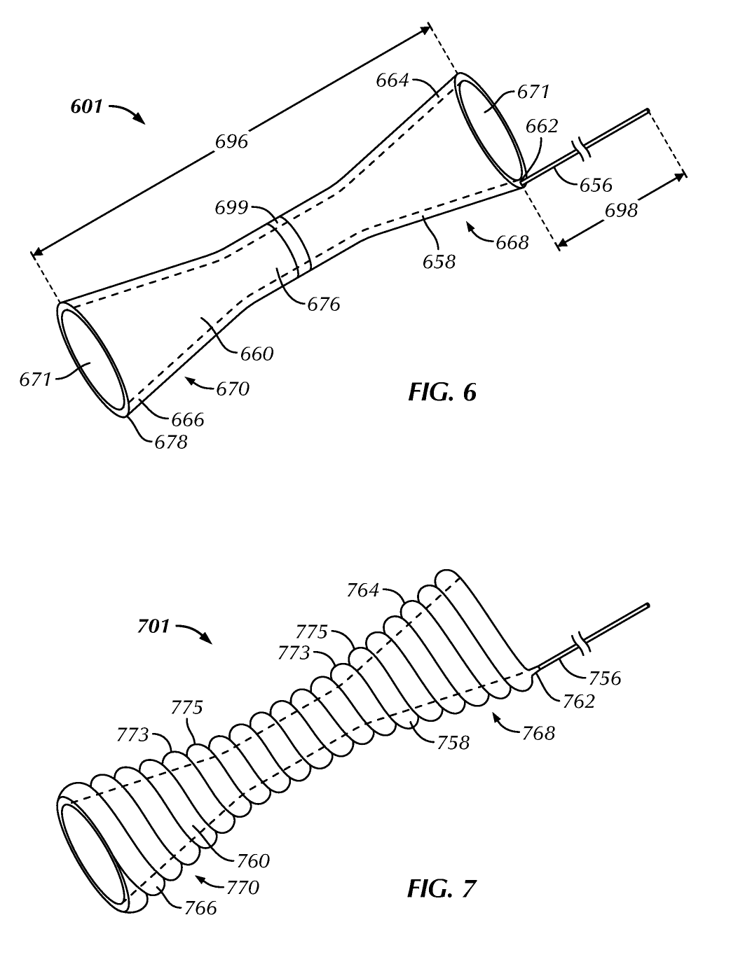

FIG. 6 illustrates in greater detail an example alignment device 601 for capturing distal ends of a guidewire and a specialty catheter advanced in a retrograde direction, and for subsequently guiding a second guidewire advanced in an antegrade direction. This alignment device 601 includes a positioning member 656 and a tubular member 658 defining a passageway 660. A distal end portion 662 of the positioning member 656 can be eccentrically coupled with the tubular member 658, and a transition between the positioning member 656 and the tubular member 658 can optionally include an angled (or skived) side opening. In an example, the positioning member 656 can be an elongated hypotube, ribbon or wire to which the tubular member 658 is fused or bonded. In various examples, a length 696 of the tubular member 658 is less than a length 698 of the positioning member 656.

The tubular member 658 can extend from a proximal end portion 664 to a distal end portion 666, each of which can include a progressive size-changing shape such as a funnel 668, 670. The funnels 668, 670 can have openings 671 facing away from one another and can be separated by an intermediate narrow neck (or reduced diameter) passageway 676. An outer surface portion 678 around the opening 671 of each funnel 668, 670 can include a diameter equal to or slightly larger than a diameter of an inner surface of a guide catheter. In an example, the diameter of the outer surface portion 678 is expandable between about 5 French (F) and about 8 F. This allows the funnels 668, 670 to engage against the inner surface of the guide catheter or guide extension catheter or an adjacent luminal wall of a blood vessel and urge received distal ends of guidewires and specialty catheters into alignment with the narrow neck passageway 676. In various examples, the narrow neck passageway 676 can be configured to receive guidewires having a diameter of 0.010 inches (in), 0.014 in, 0.018 in, 0.035 in, 0.038 in or other smaller or larger diameters.

Optionally, the tubular member 658 can include one or more radiopaque markers 699, which can help the treating clinician verify that the funnels 668, 670 are properly positioned within or partially beyond the guide catheter or guide extension catheter during use. For example, the markers 699 can help the clinician verify that the proximal funnel 668 is positioned within the guide catheter or guide extension catheter during use and the distal funnel 670 is positioned partially beyond the guide catheter or guide extension catheter, engaging against a circumferential luminal vessel wall, during use. The markers 699 can be positioned near the openings 671 of the funnels 668, 670 or around the narrow neck passageway 676.

The tubular member 658 can be made from an elastomeric material, a shape memory material and/or reinforcing fibers or wires. The elastomeric material can include, for example, a polyether block amide (e.g., PEBAX block copolymer available from Arkema, which is headquartered in Colombes, France), nylon, polytetrafluoroethylene (PTFE), polyurethane or silicone. To strengthen walls of the tubular member 658, the elastomeric material can be reinforced with fibers or wires, which can be arranged structurally in coiled or braided configurations. The funnels 668, 670 can include a self-expandable material such as nitinol, which can undergo deformations when under the influence of force and then spring back to its original shape after the force is removed. The exterior surfaces of the tubular member can be coated with a friction-reducing material to ease its movement within the guide catheter.

FIG. 7 illustrates in greater detail another example of an alignment device 701 for capturing distal ends of a guidewire and a specialty catheter advanced in a retrograde direction, and for subsequently guiding a second guidewire advanced in an antegrade direction. This alignment device 701 includes a positioning member 756 and a tubular member 758 defining a passageway 760. A distal end portion 762 of the positioning member 756 can be eccentrically coupled with the tubular member 758.

The tubular member 758 can extend from a proximal end portion 764 to a distal end portion 766, each of which can include a progressive size-changing shape such as a funnel 768, 770, and can be formed from an inflatable tube. The inflatable tube can be coiled in a helical manner around a central axis into a series of windings. Adjacent windings 773, 775 can be stacked against and bonded to each other, and an inner surface of the series of windings, when inflated, can define the passageway 760. The inflatable tube can include two different polymer tubes, one slightly smaller than the other. The smaller, inner tube can be formed from a polymer having sufficient radial stiffness to resist collapse or bursting when exposed to inflation pressures, and the larger, outer tube can be formed from a polymer configured to exhibit adhesive properties when heated. The inner and outer tubes can include polymers having different melting or softening temperatures, with the inner tube including the polymer with the higher melting temperature. The inner and outer tubes can include the same or similar polymers, with the polymer of the inner tube being cross-linked for strength and with the polymer of the outer tube not being cross-linked.

In this example, the positioning member 756 can be an elongated hypotube to which the tubular member 758 is fused or bonded. The hypotube can include a lumen for providing inflation fluid to, or withdrawing inflation fluid from, the tubular member 758. The lumen of the positioning member 756 can be in fluid communication with a manifold, couplable to an inflation syringe, at its proximal end and can be in fluid communication with the interior of the tubular member 758 near its distal end.

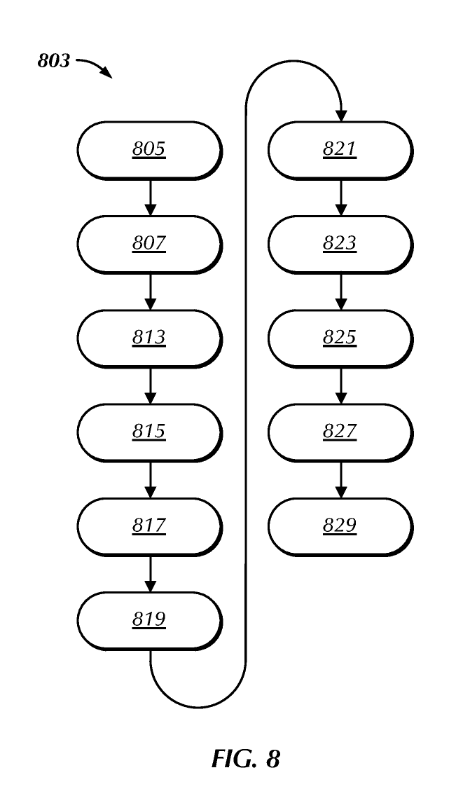

FIG. 8 illustrates an example method 803 of using an alignment device to create a bi-directional path through an occlusion. While the following method steps have been arranged and described in a certain order, such order is not intended to be limiting. One or more steps can be performed in an alternative order or not performed at all.

The method can be initiated by positioning guide catheters on each side of the occlusion. At 805, a first guide catheter can be introduced into a blood vessel at a location downstream of the occlusion and positioned for retrograde delivery of a guidewire and a specialty catheter. At 807, a second guide catheter can be introduced into the blood vessel at a location upstream of the occlusion and positioned for antegrade delivery of the alignment device. Optionally, a guide extension catheter, such as the GUIDELINER catheter available from Vascular Solutions, which is headquartered in Minneapolis, Minn., can be advanced through a lumen of the second guide catheter and have its distal end positioned near the proximal end of the occlusion.

With the guide catheters positioned within the blood vessel, various medical devices can be advanced through lumens of the guide catheters. At 813, a tubular member of the alignment device can be advanced through the lumen of the second guide catheter to a position near—within or partially beyond—the distal end of the guide catheter or the guide extension catheter. At this position, the outer surface of the proximal funnel or other progressive size-changing shape of the tubular member can engage with an inner surface of the second guide catheter or the guide extension catheter and the outer surface of the distal funnel or other progressive size-changing shape of the tubular member can engage the inner surface of the second guide catheter or the guide extension catheter or an adjacent luminal wall of the blood vessel. At 815, the guidewire and the specialty catheter can be advanced through the lumens of the first guide catheter and blood vessels toward the distal end of the occlusion. Together, the guidewire with the support of the specialty catheter can be manipulated from the distal end of the occlusion to its proximal end and, at 817, can be guided into the tubular member of the alignment device. More specifically, the guidewire can be guided into a narrow neck passageway of the tubular member and the distal end of the specialty catheter can be guided into a mating arrangement with the distal funnel such that the lumen of the specialty catheter aligns with the narrow neck passageway.

Together, the second guide catheter, the optional guide extension catheter, the tubular member of the alignment device, and the specialty catheter can form the continuous, bi-directional path between a vessel opening upstream of the occlusion and a vessel opening downstream of the occlusion. This path can be used to position a desired guidewire to further treat the occlusion. At 819, the guidewire advanced through the occlusion can be withdrawn from the blood vessel by pulling on its proximal end at a location near a valve of the first guide catheter. At 821, a new or different guidewire can be advanced in an antegrade or retrograde direction using the established bi-directional path. The new or different guidewire, if advanced in the antegrade direction, can travel through one or more of the second guide catheter and the guide extension catheter (if present), the tubular member of the alignment device in a proximal-to-distal direction, and the specialty catheter. The proximal funnel or other progressive size-changing shape of the tubular member can urge the guidewire into alignment with a lumen of the specialty catheter. The new or different guidewire, if advanced in the retrograde direction, can travel through one or more of the specialty catheter, the tubular member of the alignment device in a distal-to-proximal direction, and the guide extension catheter (if present) and the second guide catheter. In many uses, the new or different guidewire will only be advanced along the bi-directional path until its distal end crosses and is positioned beyond the occlusion site relative to its insertion site.

With the new or different guidewire in place across the occlusion site, the components forming the bi-directional path can be removed and one or more occlusion treatment devices can be inserted. At 823, the alignment device can be withdrawn from the blood vessel by pulling a positioning member attached to, and extending proximally from, the tubular member. A proximal end of the positioning member can be located near a valve of the second guide catheter. At 825, the specialty catheter can be withdrawn from the blood vessel by pulling on its proximal end located near the valve of the first guide catheter. At 827, the guide catheter that is not being utilized by the new or different guidewire can be removed and vessel sealing procedures can be performed at its site of insertion. Finally, at 829, a treatment device can be introduced into the blood vessel and, using the new or different guidewire as a rail, advanced to the occlusion site for dilatation, placement of a stent or another treatment procedure.

Closing Notes:

The present assemblies, alignment devices and methods can be used by a treating clinician to create a bi-directional path through an occlusion, particularly CTOs, using a combined antegrade and retrograde approach. The occlusion can be penetrated using a retrograde-advanced guidewire supported by a specialty catheter. A distal funnel or other progressive size-changing shape, for example, of an antegrade-advanced alignment device can capture distal end portions of the guidewire and the specialty catheter and can align portions of the bi-directional path through the occlusion. A proximal funnel or other progressive size-changing shape, for example, of the alignment device can then be used to urge a second guidewire advanced in the antegrade direction through portions of the path.

The assemblies, alignment devices and methods improve upon existing retrograde techniques, specifically guidewire capture and exchange techniques, by providing the treating clinician with more control over the guidewires used in retrograde procedures, eliminating the need for externalizing guidewires, and eliminating the need to insert a specialty catheter post-guidewire capture and externalization to effectuate a guidewire exchange. This can allow for, among other things, shorter procedure times, reduced radiation exposure for the treating clinician and patient, and less medical waste.

The above Detailed Description includes references to the accompanying drawings, which form a part of the Detailed Description. The Detailed Description should be read with reference to the drawings. The drawings show, by way of illustration, specific embodiments in which the present assemblies, alignment devices and methods can be practiced. These embodiments are also referred to herein as “examples.”

The Detailed Description is intended to be illustrative and not restrictive. For example, the above-described examples (or one or more features or components thereof) can be used in combination with each other. Other embodiments can be used, such as by one of ordinary skill in the art upon reviewing the above Detailed Description. Also, various features or components have been or can be grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment:

In Example 1, an assembly for creating a bi-directional path through an occlusion can comprise an alignment device. The alignment device can include a positioning member and a tubular member defining a passageway. A distal end portion of the positioning member can be coupled with the tubular member. A proximal end portion and a distal end portion of the tubular member can include a configuration having a progressive size-changing shape. Each size-changing shape can have an opening with a diameter greater than a diameter of an intermediate portion of the tubular member.

In Example 2, the assembly of Example 1 can optionally be configured such that the positioning member is eccentrically coupled with the tubular member.

In Example 3, the assembly of any one of Examples 1 or 2 can optionally be configured such that the proximal end portion of the tubular member includes an angled side opening.

In Example 4, the assembly of any one or any combination of Examples 1-3 can optionally be configured such that the positioning member includes an elongated hypotube, ribbon, or wire.

In Example 5, the assembly of any one or any combination of Examples 1-4 can optionally be configured such that an intermediate portion or the distal end portion of the positioning member includes an arcuate cross-sectional shape configured to cradle a guidewire.

In Example 6, the assembly of any one or any combination of Examples 1-5 can optionally be configured such that a length of the tubular member is less than a length of the positioning member.

In Example 7, the assembly of any one or any combination of Examples 1-6 can optionally be configured such that the progressive size-changing shape at the proximal end portion and the distal end portion of the tubular member is a funnel.

In Example 8, the assembly of any one or any combination of Examples 1-7 can optionally be configured such that the progressive size-changing shape at the proximal end of the tubular member defines a narrowing portion of the passageway in a proximal-to-distal direction.

In Example 9, the assembly of any one or any combination of Examples 1-8 can optionally be configured such that the progressive size-changing shape at the distal end of the tubular member defines an enlarging portion of the passageway in a proximal-to-distal direction.

In Example 10, the assembly of Example 9 can optionally be configured such that the progressive size-changing shape at the distal end of the tubular member is sized and shaped to receive a tip of a support catheter.

In Example 11, the assembly of any one of Examples 9 or 10 can optionally be configured such that the tubular member includes an hourglass shape.

In Example 12, the assembly of any one or any combination of Examples 1-11 can optionally further comprise a guide catheter having a proximal end, a distal end, a lumen extending between the proximal end and the distal end, and an inner surface, wherein the tubular member is configured to be positioned at least partially in the lumen.

In Example 13, the assembly of Example 12 can optionally be configured such that an outer surface portion of at least one progressive size-changing shape of the tubular member is engageable with the inner surface of the guide catheter.

In Example 14, the assembly of Example 13 can optionally be configured such that a diameter of the outer surface portion of each progressive size-changing shape, in a relaxed configuration, is larger than a diameter of the inner surface of the guide catheter.

In Example 15, the assembly of any one of Examples 13 or 14 can optionally be configured such that a diameter of the outer surface portion of each progressive size-changing shape is expandable at least between about 5 F and about 8 F.

In Example 16, an alignment device for use with a guide catheter can comprise a tubular member defining a passageway and a positioning member. The positioning member can be coupled at least to a proximal end portion of the tubular member and extends proximally therefrom for slidably positioning the tubular member solely within the guide catheter. A distal end portion of the tubular member can include a funnel having an opening aligned with a distal opening of the guide catheter and configured to receive a tip of a specialty catheter when positioned within or at least partially beyond the distal opening of the guide catheter.

In Example 17, the alignment device of Example 16 can optionally be configured such that a diameter of an outer surface portion of the funnel, in a relaxed configuration, is larger than an inner diameter of the guide catheter.

In Example 18, the alignment device of any one of Examples 16 or 17 can optionally be configured such that a proximal end portion of the tubular member includes a second funnel having an opening facing away from the opening of the funnel at the distal end portion of the tubular member.

In Example 19, the alignment device of any one or any combination of Examples 16-18 can optionally be configured such that the positioning member is eccentrically coupled with the proximal end portion of the tubular member.

In Example 20, a method for creating a bi-directional path through an occlusion can include introducing a first guide catheter into a blood vessel at a location upstream of the occlusion and advancing the first guide catheter in an antegrade direction toward the occlusion's proximal end. An alignment device, including a positioning member and a tubular member having bi-directional proximal and distal funnels separated by a narrow neck passageway, can be introduced into a proximal end portion of the first guide catheter and advanced to or partially beyond the first guide catheter's distal end portion, where the proximal and distal funnels can engage against an inner surface of the first guide catheter or an adjacent luminal wall of the blood vessel. A first guidewire and a specialty catheter can be introduced into the blood vessel at a location downstream of the occlusion and advanced in a retrograde direction toward the occlusion's distal end. With the first guidewire leading the way, the first guidewire and the specialty catheter can be manipulated through the occlusion and into the distal funnel of the tubular member.

In Example 21, the method of Example 20 can optionally be configured such that manipulating the first guidewire and the specialty catheter through the occlusion includes creating a continuous channel between proximal and distal ends of the occlusion.

In Example 22, the method of any one of Examples 20 or 21 can optionally be configured such that manipulating the first guidewire and the specialty catheter into the distal funnel of the tubular member includes aligning a lumen of the specialty catheter and the narrow neck passageway of the tubular member.

In Example 23, the method of any one or any combination of Examples 20-22 can optionally further comprise withdrawing the first guidewire from the blood vessel from the location downstream of the occlusion, and advancing a second guidewire, introduced into the blood vessel from the location upstream of the occlusion, through the narrow neck passageway of the tubular member and into the lumen of the specialty catheter.

In Example 24, the method of any one or any combination of Examples 20-23 can optionally further comprise withdrawing the specialty catheter from the blood vessel from the location downstream of the occlusion, and withdrawing the alignment device from the blood vessel from the location upstream of the occlusion.

In Example 25, the method of Example 24 can optionally further comprise introducing a treatment device into the first guide catheter and, using the second guidewire as a rail, advancing the treatment device in the antegrade direction to the occlusion.

In Example 26, the method of any one or any combination of Examples 20-25 can optionally further comprise introducing a second guide catheter into the blood vessel at the location downstream of the occlusion, and using a lumen of the second guide catheter to advance the first guidewire and the specialty catheter.

In Example 27, the method of any one or any combination of Examples 20-26 can optionally further comprise advancing a guide extension catheter through the first guide catheter in the antegrade direction to a position near the proximal end of the occlusion, thereby creating an extension to the first guide catheter. In this scenario, advancing the tubular member to or partially beyond the distal end portion of the first guide catheter can include allowing the proximal funnel to engage against an inner surface of the guide extension catheter and allowing the distal funnel to engage against the inner surface of the guide extension catheter or the adjacent luminal wall.

In Example 28, the assembly, device or method of any one or any combination of Examples 1-27 can optionally be configured such that all features, components, operations or other options are available to use or select from.

Certain terms are used throughout this patent document to refer to particular features or components. As one skilled in the art will appreciate, different persons may refer to the same feature or component by different names. This patent document does not intend to distinguish between components or features that differ in name but not in function.

For the following defined terms, certain definitions shall be applied unless a different definition is given elsewhere in this patent document. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” The terms “clinician” or “treating clinician” refer to a doctor, nurse or other care provider and can include support personnel. The terms “distal” and “proximal” are used to generally refer to a position or direction relative to a treating clinician. “Distal” or “distally” refer to a position that is further from where the treating clinician manipulates or controls a device. Similarly, “advance” or “advancing” refer to a direction away from the treating clinician. “Proximal” and “proximally” refer to a position that is closer to where the treating clinician manipulates or controls the device. Similarly, “retract,” “retracting,” “withdraw,” “withdrawing,” or “removing” refer to a direction toward the treating clinician. The term “patient” refers to a human patient or an animal patient. Finally, the term “specialty catheter” refers to a micro-catheter or a support catheter used to provide support to, or exchange of, a guidewire.

The scope of the invention should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, an assembly, kit or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects. It is to be understood that although dependent claims may be set out in single dependent form, the features of these claims can be combined as if the claims were in multiple dependent form.

The Abstract is provided to allow the reader to quickly ascertain the nature of the technical disclosure. It is submitted with the understanding that it will not be used to interpret or limit the scope or meaning of the claims.

What is claimed is:

1. An assembly for creating a bi-directional path through an occlusion, comprising:
an alignment device including a positioning member and a tubular member defining a passageway,
a distal end portion of the positioning member coupled with the tubular member,
a proximal end portion and a distal end portion of the tubular member including a configuration having a progressive size-changing shape, each size-changing shape having an opening with a diameter greater than a diameter of an intermediate portion of the tubular member.

2. The assembly of claim 1, wherein the positioning member is eccentrically coupled with the tubular member.

3. The assembly of claim 1, wherein the proximal end portion of the tubular member includes an angled side opening.

4. The assembly of claim 1, wherein the positioning member includes an elongated hypotube, ribbon, or wire.

5. The assembly of claim 1, wherein an intermediate portion or the distal end portion of the positioning member includes an arcuate cross-sectional shape configured to cradle a guidewire.

6. The assembly of claim 1, wherein the progressive size-changing shape at the proximal end portion and the distal end portion of the tubular member is a funnel.

7. The assembly of claim 1, wherein the progressive size-changing shape at the proximal end of the tubular member defines a narrowing portion of the passageway in a proximal-to-distal direction.

8. The assembly of claim 7, wherein the progressive size-changing shape at the distal end of the tubular member defines an enlarging portion of the passageway in a proximal-to-distal direction.

9. The assembly of claim 8, wherein the progressive size-changing shape at the distal end of the tubular member is sized and shaped to receive a tip of a specialty catheter.

10. The assembly of claim 1, further comprising a guide catheter having a proximal end, a distal end, a lumen extending between the proximal end and the distal end, and an inner surface, wherein the tubular member is configured to be positioned at least partially in the lumen; and
wherein an outer surface portion of at least one progressive size-changing shape of the tubular member is engageable with the inner surface of the guide catheter.

11. The assembly of claim 10, wherein a diameter of the outer surface portion of each progressive size-changing shape, in a relaxed configuration, is larger than a diameter of the inner surface of the guide catheter.

12. The assembly of claim 10, wherein a diameter of the outer surface portion of each progressive size-changing shape is expandable.

13. An alignment device for use with a guide catheter, comprising:
a tubular member defining a passageway; and
a positioning member eccentrically coupled at least to a proximal end portion of the tubular member and extending proximally therefrom for slidably positioning the tubular member within the guide catheter,
a distal end portion of the tubular member including a funnel having an opening for alignment with a distal opening of the guide catheter and configured to receive a tip of a specialty catheter when positioned within or at least partially beyond the distal opening of the guide catheter.

14. The alignment device of claim 13, wherein a diameter of an outer surface portion of the funnel, in a relaxed configuration, is larger than an inner diameter of the guide catheter.

15. The alignment device of claim 13, wherein a proximal end portion of the tubular member includes a second funnel having an opening facing away from the opening of the funnel at the distal end portion of the tubular member.

16. A method, comprising:
advancing a first guide catheter, introduced into a blood vessel at a location upstream of an occlusion, in an antegrade direction toward a proximal end of the occlusion;
advancing an alignment device, including a positioning member and a tubular member having bi-directional proximal and distal funnels separated by a narrow neck passageway, through the first guide catheter such that the distal funnel is near or partially beyond a distal end portion of the first guide catheter, including allowing the proximal funnel to engage against an inner surface of the first guide catheter and allowing the distal funnel to engage against the inner surface of the first guide catheter or an adjacent luminal wall of the blood vessel; and
advancing a first guidewire and a specialty catheter, introduced into the blood vessel at a location downstream of the occlusion, in a retrograde direction, including manipulating the first guidewire and the specialty catheter toward and through the occlusion and into the distal funnel of the tubular member.

17. The method of claim 16, wherein manipulating the first guidewire and the specialty catheter through the occlusion includes creating a continuous channel between proximal and distal ends of the occlusion.

18. The method of claim 16, wherein manipulating the first guidewire and the specialty catheter into the distal funnel of the tubular member includes aligning a lumen of the specialty catheter and the narrow neck passageway of the tubular member.

19. The method of claim 18, further comprising withdrawing the first guidewire from the blood vessel from the location downstream of the occlusion, and advancing a second guidewire, introduced into the blood vessel from the location upstream of the occlusion, through the narrow neck passageway of the tubular member and into the lumen of the specialty catheter.

20. The method of claim 19, further comprising withdrawing the specialty catheter from the blood vessel from the location downstream of the occlusion, and withdrawing the alignment device from the blood vessel from the location upstream of the occlusion.

21. The method of claim 20, further comprising introducing a treatment device into the first guide catheter and, using the second guidewire as a rail, advancing the treatment device in the antegrade direction to the occlusion.

22. The method of claim 16, further comprising introducing a second guide catheter into the blood vessel at the location downstream of the occlusion, and using a lumen of the second guide catheter to advance the first guidewire and the specialty catheter.

23. The method of claim 16, further comprising:
advancing a guide extension catheter through the first guide catheter in the antegrade direction to a position near the proximal end of the occlusion, thereby creating an extension to the first guide catheter, and
wherein advancing the alignment device through the first guide catheter such that the distal funnel is near or partially beyond the distal end portion of the first guide catheter includes allowing the proximal funnel to engage against an inner surface of the guide extension catheter and allowing the distal funnel to engage against the inner surface of the guide extension catheter or the adjacent luminal wall.
